(19) United States
(12) Patent Application Publication
(10) Pub. No.: US 2022/0273383 A1
(43) Pub. Date: Sep. 1, 2022

(54) DEVICE FOR PROPELLING AND STEERING A MICROSTRUCTURE

(71) Applicant: ROBEAUTE, Paris (FR)

(72) Inventors: Bertrand DUPLAT, Paris (FR); Ali OULMAS, Paris (FR); Quentin FRANCOIS, Paris (FR)

(73) Assignee: ROBEAUTE, Paris (FR)

(21) Appl. No.: 17/761,847

(22) PCT Filed: Sep. 18, 2020
(86) PCT No.: PCT/FR2020/051623

§ 371 Date: Mar. 18, 2022

(30) Foreign Application Priority Data
Sep. 20, 2019 (FR) 1910368

(51) Int. Cl. A61B 34/00; H01F 7/122; F16F 1/36; F16F 1/373

(52) U.S. Cl. CPC A61B 34/73; H01F 7/122; F16F 1/3605; F16F 1/373; A61B 2017/00345

(57) ABSTRACT

This device includes a propulsion element including at least one portion deformable in elongation/contraction according to a main axis (X₂) connecting a front portion and a rear portion. At least two guide elements adapted to generate, under the effect of an energy supply, a rotation of the propulsion element respectively about a first axis of rotation and about a second axis of rotation transverse to each other and to the main axis (X₂) of the propulsion element. A control unit configured to actuate a rotation of the propulsion element about at least one axis transverse to the main axis (X₂) in a coordinated manner with a deformation of the deformable element of the propulsion element in elongation/contraction according the main axis (X₂).

### TECHNICAL FIELD

The present invention relates to a device for propelling and steering a microstructure, for example a mobile flexible tube such as a stent or a catheter, or still a microrobot, intended to move in a fluid, in particular in a vessel of a subject, such as an artery or a vein, or in an organ of a subject, such as a brain, a heart, a liver, a pancreas, etc. A mobile flexible tube or a microrobot could be used to perform various biomedical operations, in particular in the context of minimally-invasive surgeries or targeted therapies.

### PRIOR ART

The possibility of reaching deep and functional structures without creating any damage is a major challenge in minimally-invasive surgery, in particular in neurosurgery. Thanks to microtechnologies, it has become possible to send a completely autonomous micro-medical device inside a vessel or an organ of a subject. Nonetheless, such a micro-medical device requires a system enabling propulsion and steering thereof in three dimensions with an accuracy at least equal to the size of the device, even in a heterogeneous and sensitive environment.

In this context, the invention aims to provide a device for propelling and steering a microstructure, such as a flexible tube or a microrobot, ensuring effective and reliable propulsion and steering of the microstructure, including in a fluid environment with a low Reynolds number, with an accuracy at least equal to the size of the microstructure, while preserving as much as possible the integrity of the environment in which the microstructure moves.

### DESCRIPTION OF THE INVENTION

To this end, an object of the invention a device for propelling and steering a microstructure, such as a flexible tube or a microrobot, comprising:
a propulsion element including at least one portion deformable in elongation/contraction according to a main axis connecting a front portion and a rear portion of the propulsion element;
at least two guide elements adapted to generate, under the effect of an energy supply by a respective connection to an energy source, a rotation of the propulsion element respectively about a first axis of rotation and about a second axis of rotation transverse to each other and to the main axis of the propulsion element;
a control unit configured to actuate, by selectively controlling one or more of the connections to an energy source, a rotation of the propulsion element about at least one axis transverse to the main axis in a coordinated manner with a deformation of the deformable portion of the propulsion element in elongation/contraction according to the main axis, the guide elements further comprising at least two guide segments based on an active material reversibly deformable under the effect of an energy supply by a respective connection to an energy source, each guide segment being adapted to generate by the deformation thereof, under the effect of an energy supply, a rotation of the propulsion element about an axis of rotation transverse to the main axis of the propulsion element.

A propulsion and steering device according to the invention allows steering the microstructure in three dimensions, thanks to the possibility of actuating a rotation of the propulsion element about at least two axes of rotation, transverse to each other and to the main axis, in a coordinated manner with a propulsion of the microstructure obtained by a deformation of the deformable portion of the propulsion element. In the context of the invention, two axes are transverse to each other when they are not parallel, which includes the case of two axes perpendicular to each other, yet without being limited thereto.

In the context of the invention, the actuation of the rotation is carried out in a coordinated manner, in particular simultaneously or sequentially, with the deformation of the deformable portion of the propulsion element, in order to obtain desired movement and trajectory of the microstructure in the environment within which it moves, which is in particular a fluid environment with a low Reynolds number. More specifically, the actuation of the rotation could be carried out simultaneously with the deformation of the deformable portion of the propulsion element, or sequentially with the deformation of the deformable portion of the propulsion element, i.e. such that the rotation and the deformation are performed one after another, in particular in a repetitive manner.

In the context of the invention, a microstructure provided with the propulsion and steering device according to the invention typically has an external diameter smaller than or equal to 5 mm, in particular smaller than or equal to 2 mm or 1 mm.

According to one feature, the propulsion element comprises at least a first guide segment and a second guide segment such that the deformation of the first guide segment generates a rotation of the propulsion element about a first axis of rotation perpendicular to the main axis of the propulsion element, and the deformation of the second guide segment generates a rotation of the propulsion element about a second axis of rotation perpendicular to both the main axis of the propulsion element and to the first axis of rotation.

According to an embodiment of a guide segment, the segment is an area of the deformable portion of the propulsion element which is coated with the active material. According to another embodiment of a guide segment, the segment is a segment comprising a support provided with the active material which is attached on the deformable portion of the propulsion element.

According to one embodiment, the deformable portion of the propulsion element is made of a material having a Young's modulus comprised in a range from 0.1 to 10 GPa, preferably from 0.5 to 2 GPa. In one embodiment, the front portion, the rear portion and the deformable portion of the propulsion element are all made of the same material. In one embodiment, the constituent material of the front portion, the rear portion and the deformable portion is a biocompatible polymer. An example of a suitable material for the front portion, the rear portion and/or the deformable portion is a UV-curable hybrid inorganic-organic polymer, such as the product ORMOCLEAR manufactured by the company MICRO RESIST TECHNOLOGY GmbH.

In one embodiment, at least one guide segment includes an electroactive material or a bimetallic element, the propulsion and steering device comprising an electrical energy source connected to the guide segment so as to activate the deformation thereof. In particular, the energy source is an electrical power supply connected by an electrical wire or cable to the electroactive material or to the bimetallic element of the guide segment.

In the context of the invention, an electroactive material is a material which deforms, in particular by changing its shape or its size, under the effect of an electrical energy supply. Examples of suitable electroactive materials in the context of the invention include shape memory alloys such as Nitinol; or electroactive polymers (EAP), in particular dielectric electroactive polymers and ionic electroactive polymers. As a non-limiting example, an ionic electroactive polymer that could be used in the context of the invention is poly(3,4-ethylenedioxythiophene) (PEDOT).

In the context of the invention, a bimetallic element is an element including two materials which, under the effect of a heat supply, which could be induced in particular by an electric current when the materials are electrically conductive, deform elastically individually according to different mechanical characteristics, which creates, by their solid contact, a very significant deformation of the bimetal element. Such bimetallic elements could be formed, in particular, by co-rolling two metal strips. Examples of suitable bimetallic elements in the context of the invention are copper and steel bimetals, or iron and nickel bimetals, as these are bimetals combining metallic materials having quite different thermal expansion coefficients.

In one embodiment, at least one guide segment includes a photoactive material, the propulsion and steering device comprising a radiation source whose radiation is emitted opposite the guide segment so as to activate the deformation thereof. In particular, the radiation source is a laser source or a LED (light-emitting diode) whose radiation is transmitted up to the photoactive material of the guide segment using an optical fibre having a distal end positioned opposite the photoactive material of the guide segment.

In the context of the invention, a photoactive material is a material which deforms under the effect of a radiation, in particular under the effect of a light energy supply. Examples of suitable photoactive materials in the context of the invention comprise networks of liquid crystals comprising azobenzene molecules. The radiation source could then be a white light source, comprising all wavelengths of the visible spectrum. As a non-limiting example, a photoactive material that could be used in the context of the invention is an actuator based on double photo-sensitive liquid crystals, containing in particular an azomerocyanine dye locally converted into the hydroxyazopyridinium form by acid treatment.

According to one feature, at least two of said guide segments are configured to actuate a deformation of the deformable portion of the propulsion element in elongation/contraction according to the main axis when they are deformed simultaneously and to actuate a rotation of the propulsion element about an axis of rotation transverse to the main axis when they are selectively deformed. By selectively supplying energy to the guide segments, it is then possible to actuate a rotation and a deformation in elongation/contraction of the propulsion element, which allows ensuring directional piloting and propulsion of the microstructure.

According to one feature, the guide segments are distributed isotropically around the main axis of the propulsion element. This results in an improved control for the directional steering of the propulsion element.

According to one embodiment, the deformable portion of the propulsion element comprises a unique flexible leg helically disposed around the main axis between the front portion and the rear portion of the propulsion element, the flexible leg including at least two guide segments distributed along the flexible leg and configured to generate by their deformation a rotation of the propulsion element respectively about a first axis of rotation and about a second axis of rotation transverse to each other and to the main axis of the propulsion element.

According to another embodiment, the deformable portion of the propulsion element comprises at least two flexible legs helically disposed around the main axis between the front portion and the rear portion of the propulsion element, the propulsion and steering device including at least one pair of guide segments, respectively on a first flexible leg and on a second flexible leg, configured to generate by their deformation a rotation of the propulsion element respectively about a first axis of rotation and about a second axis of rotation transverse to each other and to the main axis of the propulsion element.

According to an aspect of the invention, the guide elements comprise at least two electromagnetic guide coils, each provided with a respective connection to an electrical energy source, which form an electromagnetic transducer with a magnet secured to the propulsion element, the magnet being substantially parallel to the main axis of the propulsion element in the rest position, each guide coil being adapted to generate, under the effect of an electrical energy supply, a rotation of the magnet with respect to its rest position causing a rotation of the propulsion element about an axis of rotation transverse to the main axis of the propulsion element.

According to one feature, for each electromagnetic transducer comprising a guide coil and the magnet secured to the propulsion element, the magnet is inserted inside the guide coil for the actuation of a rotation of the propulsion element. Such an arrangement ensures electromagnetic conversion efficiency allowing controlling the rotation of the propulsion element reliably and accurately by acting on the electrical connection of each guide coil. Of course, the polarity of the magnet and the power supply of each guide coil are adapted in order to obtain the desired rotation of the propulsion element.

According to one embodiment, the propulsion and steering device further comprises a linear actuation electromagnetic coil, provided with a respective connection to an electrical energy source, which also forms an electromagnetic transducer with the magnet secured to the propulsion element, the linear actuation coil being adapted to generate, under the effect of an electrical energy supply, a translation of the magnet parallel to the main axis causing deformation of the deformable portion of the propulsion element in elongation/contraction according to the main axis. By selectively powering the guide coils and the linear actuation coil electrically, it is then possible to actuate a rotation and a deformation in elongation/contraction of the propulsion element, which allows ensuring directional steering and propulsion of the microstructure.

According to another embodiment, at least two of said electromagnetic guide coils are configured to actuate a deformation of the deformable portion of the propulsion element in elongation/contraction according to the main axis when they are simultaneously powered with electrical energy and to generate a rotation of the magnet with respect to its rest position causing a rotation of the propulsion element about an axis of rotation transverse to the main axis when they are selectively powered. By selectively powering the guide coils electrically, for example simultaneously or successively, it is then possible to actuate a rotation and a deformation in elongation/contraction of the propulsion element, which allows ensuring directional steering and propulsion of the microstructure.

According to one embodiment, each guide coil has its central axis substantially parallel to the main axis of the propulsion element. According to another embodiment, each guide coil has its central axis substantially perpendicular to the main axis of the propulsion element.

The number of guide coils may be any number greater than or equal to two. In particular, in a non-limiting manner, the following arrangements could be considered in the context of the invention: two guide coils disposed one behind another according to the direction of the main axis of the propulsion element, with their central axes not coincident with and substantially parallel to the main axis of the propulsion element; two guide disposed arranged side-by-side with their central axes substantially parallel to the main axis of the propulsion element; at least three guide coils, in particular three, four, five or six guide coils, disposed one behind another according to the direction of the main axis of the propulsion element, with their central axes not coincident and substantially parallel to the main axis of the propulsion element; at least three guide coils, in particular three, four, five or six guide coils, distributed around the main axis of the propulsion element, with their central axes substantially parallel to the main axis of the propulsion element; at least three guide coils, in particular three, four, five or six guide coils, distributed around the main axis of the propulsion element, with their central axes substantially perpendicular to the main axis of the propulsion element.

According to one feature, the control unit is further configured to actuate a deformation of the deformable portion of the propulsion element by elongation/contraction according to the main axis. Thus, it is possible to optimally coordinate the actuation of the rotation of the propulsion element and the actuation of the elongation/contraction deformation of the propulsion element.

According to one feature, the propulsion and steering device comprises a linear actuator configured to actuate a deformation of the deformable portion of the propulsion element in elongation/contraction according to the main axis. According to one embodiment, the linear actuator comprises an electromagnetic transducer including a combination of an electromagnetic coil fastened to one end of the deformable portion and a permanent magnet fastened to the other end of the deformable portion. According to one embodiment, the linear actuator comprises a pump. This embodiment is suitable in the case where the deformable portion of the propulsion element could contain a fluid in its internal volume, in particular in the case where the deformable portion has an envelope forming a continuous peripheral wall. In one embodiment, the deformable portion of the propulsion element comprises a bellow and the actuator comprises a pump.

In one embodiment, the propulsion and steering device comprises at least one propulsion cilium secured to the front portion of the propulsion element, one end of the propulsion cilium being secured to the front portion whereas the other end of the propulsion cilium is a free end configured to move freely so as to generate a non-reciprocal movement of the microstructure, in particular in a fluid with a low Reynolds number comprised between 10⁻⁵ and 10⁻¹. Thanks to the presence of such cilia, a propulsion movement of the microstructure is obtained even in a viscous or viscoelastic material, in particular in an organ of a subject such as the brain. The successive elongation/contraction cycles of the deformable portion of the propulsion element cause a movement of the propulsion cilia in the viscous or viscoelastic material, thus inducing a net propulsive force due to the interaction of the propulsion cilia with the viscous or viscoelastic material.

According to one feature, for each elongation/contraction deformation cycle of the deformable portion of the propulsion element according to its main axis, the pathway of the free end of the or each propulsion cilium in the contraction phase of the propulsion element, in a fluid with a low Reynolds number comprised between 10⁻⁵ and 10⁻¹, is different from the pathway of the free end of the or each propulsion cilium in said fluid in the elongation phase of the propulsion element. Such an implementation of the propulsion cilia with respect to the elongation and contraction phases of the deformable portion allows obtaining a non-reciprocal movement of the microstructure, which allows for an efficient movement within fluidic materials with a low Reynolds number.

In particular, in non-limiting embodiments, the pathway of the free end of the propulsion cilium/cilia in a viscous or viscoelastic material is topologically equivalent to an elliptical pathway or to a circular pathway for each cycle of elongation/contraction of the deformable portion. It should be noted that a pathway of the free end topologically equivalent to a line segment is not suitable for obtaining a non-reciprocal movement of the microstructure, even if different dynamics are applied along the pathway.

According to one embodiment, the rear portion of the propulsion element comprises at least one propulsion cilium. In the context of the invention, it should be understood that the presence of propulsion cilia only on the front portion of the propulsion element is enough. However, an arrangement with propulsion cilia also provided on the rear portion could contribute to the propulsion of the microstructure in a viscous or viscoelastic material. According to one embodiment, when the rear portion of the propulsion element includes at least one propulsion cilium at its surface, the or each propulsion cilium of the rear portion may be identical to or different from the propulsion cilium/cilia of the front portion of the propulsion element.

According to one embodiment, the or each propulsion cilium of the front portion and/or of the rear portion of the propulsion element is made of a material having a Young's modulus comprised in a range from 0.1 to 10 GPa, preferably from 0.5 to 2 GPa. According to one embodiment, the or each propulsion cilium is made of the same material as the deformable portion of the propulsion element. In one embodiment, the material of the propulsion cilium is a biocompatible polymer. Examples of materials suitable for the propulsion cilia(s) comprise polydimethylsiloxane (PDMS), silicon, or a UV-curable hybrid inorganic-organic polymer such as ORMOCLEAR.

According to one embodiment, the at least two guide elements are positioned radially outside the deformable portion.

According to one embodiment, the deformable portion includes an oscillating disc disposed between the front portion and the rear portion, the at least two guide elements being disposed between the rear portion and the oscillating disc.

According to one embodiment, the propulsion and steering device comprises at least two propulsion elements arranged one behind another, the control unit being configured to actuate cycles of deformation of the propulsion elements in elongation/contraction according to their main axes according to predefined time sequences, so as to generate a non-reciprocal movement of the microstructure, in particular in a fluid with a low Reynolds number comprised between 10⁻⁵ and 10⁻¹. Such an arrangement is another way to obtain a non-reciprocal movement of the microstructure, allowing for an efficient movement in fluids with a low Reynolds number. This arrangement could be used alone or in combination with at least one propulsion cilium to generate a non-reciprocal movement as described hereinabove.

Another object of the invention is a microstructure comprising a propulsion and steering device as described hereinabove. According to an aspect of the invention, the microstructure is configured to move in a fluidic material with a low Reynolds number, in particular a fluidic material having a Reynolds number Re comprised between 10⁻⁵ and 10⁻¹. In a known manner, the Reynolds number Re is a dimensionless quantity that quantifies the relative magnitude of the inertial forces and the viscous forces for given flow conditions. The Reynolds number Re could be expressed by the ratio between the inertial forces and the viscous forces in a fluid:

$$Re = \frac{u L}{v} ,$$

where u is the average velocity of the fluid relative to the object, L is a characteristic linear dimension, v is the kinematic viscosity of the fluid.

Another object of the invention is a method for propelling and steering a microstructure, such as a flexible tube or a microrobot, comprising a propulsion and steering device as described hereinabove, the method comprising steps wherein:
the microstructure is introduced comprising the propulsion and steering device in a fluid with a low Reynolds number in particular comprised between 10⁻⁵ and 10⁻¹;
one or more of the connections to an energy source are actuated, by selectively controlling using the control unit, a rotation of the propulsion element about at least one axis transverse to the main axis of the propulsion element in a coordinated manner with a deformation of the deformable portion of the propulsion element in elongation/contraction according to the axis main.

### BRIEF DESCRIPTION OF THE FIGURES

The features and advantages of the invention will appear in the following description of several embodiments of a device and a method for propelling and steering a microstructure according to the invention, provided only as example and made with reference to the appended drawings wherein:

FIG. 1 is a schematic section of a microrobot comprising a propulsion and steering device according to a first embodiment of the invention, with a propulsion element in the form of a helical spring with three flexible legs, each flexible leg comprising a guide segment based on an electroactive material provided with a respective electrical connection;

FIG. 2 is a section similar to FIG. 1 showing the activation of a rotational movement of the microrobot;

FIG. 3 is a partial perspective view on a larger scale of the propulsion element of the microrobot of FIGS. 1 and 2;

FIG. 4 is a section similar to FIG. 2 for a microrobot comprising a propulsion and steering device according to a second embodiment of the invention, with a propulsion element in the form of a helical spring with three flexible legs, each flexible leg comprising a guide segment based on a photoactive material associated to an optical fibre transmitting a respective radiation;

FIG. 5 is a section similar to FIG. 2 for a microrobot comprising a propulsion and steering device according to a third embodiment of the invention, with a propulsion element in the form of a helical spring with two flexible legs, each flexible leg comprising a plurality of guide segments based on an electroactive material, where each guide segment of each flexible leg is provided with a respective electrical connection so as to be able to be powered independently by an electrical power supply;

FIG. 6 is a section similar to FIG. 2 for a microrobot comprising a propulsion and steering device according to a fourth embodiment of the invention, with a propulsion element in the form of a helical spring with a unique flexible leg comprising a plurality of guide segments based on an electroactive material, where each guide segment of the flexible leg is provided with a respective electrical connection so as to be able to be powered independently by an electrical power supply;

FIG. 7 is a section similar to FIG. 2 for a microrobot comprising a propulsion and steering device according to a fifth embodiment of the invention, with two propulsion elements arranged one behind another, the control unit being configured to actuate cycles of deformation of the propulsion elements in elongation/contraction according to their main axes according to predefined temporal sequences so as to generate a non-reciprocal movement of the microstructure;

FIG. 8 is a section similar to FIG. 1 for a microrobot comprising a propulsion and steering device according to a sixth embodiment of the invention, with a propulsion element in the form of a helical spring with three flexible legs and an electromagnetic transducer with three coils comprising a linear actuation coil and two rotation guide coils each provided with a respective electrical connection;

FIG. 9 is a section similar to FIG. 8 showing the activation of a rotational movement of the microrobot;

FIG. 10 is a perspective view on a larger scale of a portion of the propulsion element of the microrobot of FIGS. 8 and 9;

FIG. 11 is a perspective view similar to FIG. 10 of a portion of the propulsion element of a microrobot comprising a propulsion and steering device according to a seventh embodiment of the invention;

FIG. 12 is a perspective view similar to FIG. 10 of a portion of the propulsion element of a microrobot comprising a propulsion and steering device according to an eighth embodiment of the invention;

FIG. 13 is a partial perspective view similar to FIG. 3 of the propulsion element of a microrobot comprising a propulsion and steering device according to a ninth embodiment of the invention;

FIG. 14 is a partial perspective view similar to FIG. 13 of the propulsion element of a microrobot comprising a propulsion and steering device according to a tenth embodiment of the invention;

FIG. 15 is a partial perspective view similar to FIG. 13 of the propulsion element of a microrobot comprising a propulsion and steering device according to an eleventh embodiment of the invention;

FIG. 16 is a partial perspective view similar to FIG. 15 of the same embodiment of the invention, but in action.

### DESCRIPTION OF EMBODIMENTS

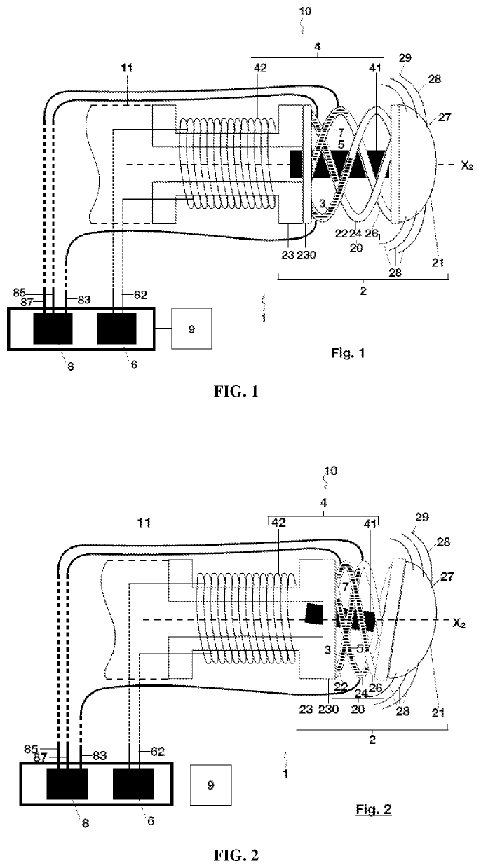

In the first embodiment represented in FIGS. 1 to 3, the microrobot 10 is configured to move in a viscous or viscoelastic material, for example in the cerebrospinal fluid or the extracellular matrix of a subject's brain, which are fluidic materials with a low Reynolds number for the microrobot.

The microrobot 10 comprises a propulsion and steering device 1 according to the invention, to which is fastened an active portion 11 of the microrobot which could, for example, be: a sensor; an actuator; a reservoir adapted to release a drug; etc.

As clearly shown in FIGS. 1 and 2, the propulsion and steering device 1 comprises a propulsion element 2 including a front portion 21, a rear portion 23, and a deformable portion 20 connecting the front portion 21 and the rear portion 23. In this first embodiment, the deformable portion 20 is a helical spring deformable in elongation/contraction according to a main axis X₂ of the propulsion element 2. The main axis X₂ of the propulsion element 2 is herein defined as a central axis of the deformable portion 20 substantially perpendicular to the plane of a distal plate 230 of the rear portion 23, to which the deformable portion 20 is fastened.

The helical spring forming the deformable portion 20 comprises three flexible legs 22, 24, 26 helically disposed around the main axis X₂ between the front portion 21 and the rear portion 23 of the propulsion element. Each flexible leg 22, 24, 26 is provided with a respective guide segment 3, 5, 7 based on an electroactive material, for example a PEDOT ionic electroactive polymer. Each of the three guide segments 3, 5, 7 is reversibly deformable under the effect of an electrical energy supply, and connected to an electrical power supply 8 by a respective electrical cable 83, 85, 87.

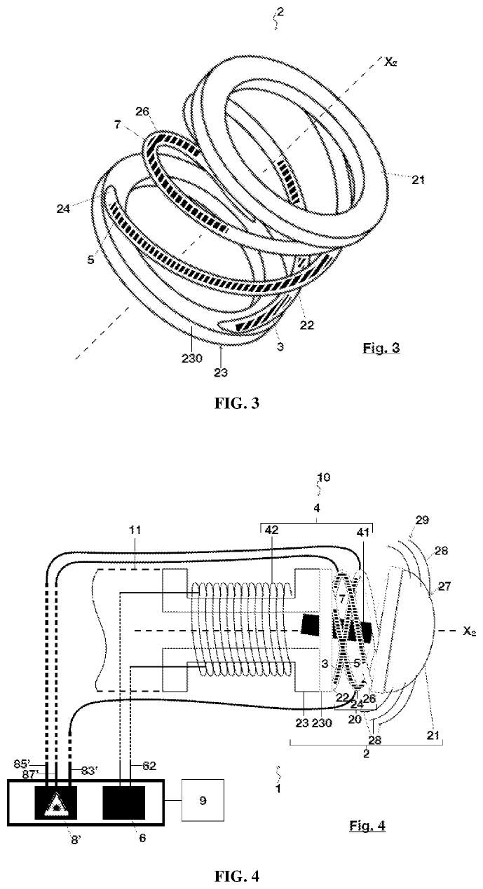

Each guide segment 3, 5, 7 is adapted to generate, by the deformation thereof when it is powered with electrical energy, a deformation of the corresponding flexible leg and a rotation of the propulsion element 2. For each guide segment 3, 5, 7, the axis of the rotation generated by the deformation of the guide segment is transverse to the main axis X₂ of the propulsion element as well as to the axes of the rotations generated by the deformation of each of the other two guide segments. As clearly shown on the larger scale view of FIG. 3, the guide segments 3, 5, 7 are isotropically distributed around the main axis X₂ of the propulsion element 2, which allows optimising the directional steering of the propulsion element. Thus, in the present invention, the guide segments 3, 5, 7 form, with the flexible legs 22, 24, 26, a unique versatile functional group ensuring both rotation and propulsion. The present invention does not feature any coupling of different elements, each ensuring a distinct function.

The propulsion and steering device 1 also comprises a linear actuator 4 configured to sequentially actuate elongation/contraction cycles of the deformable portion 20 of the propulsion element 2. The actuator 4 is an electromagnetic transducer comprising a permanent magnet 41 and an electromagnetic coil 42. The magnet 41 is fastened to the front portion 21 of the propulsion element 2, at the front end of the deformable portion 20, whereas the coil 42 is mounted on the rear portion 23, and therefore fastened to the end rear of the deformable portion 20. Depending on the electric current applied to the coil 42, the magnet 41 gets close or away from the coil 42, which induces a contraction or an elongation of the deformable portion 20.

As shown in FIGS. 1 and 2, the front portion 21 of the propulsion element 2 comprises at its surface a plurality of propulsion cilia 28, configured to interact with the material in which the microrobot 10 moves. The sequential elongation/contraction cycles of the deformable portion 20 actuated by the electromagnetic transducer 4 cause a movement of the propulsion cilia 28 in the material, producing a propulsive force which causes a movement of the microrobot 10.

For each elongation/contraction cycle of the deformable portion 20 actuated by the electromagnetic transducer 4, each propulsion cilium 28 is configured such that the pathway of the free end 29 of the propulsion cilium 28 in a viscous or viscoelastic material in the contraction phase of the deformable portion 20 is different from that of the free end 29 in the viscous or viscoelastic material in the elongation phase of the deformable portion 20. Advantageously, the pathway of the free end 29 of the propulsion cilium 28 in a viscous or viscoelastic material is topologically equivalent to an elliptical pathway or a circular pathway for each elongation/contraction cycle. A non-reciprocal movement of the microrobot 10 is thus obtained, allowing for an efficient movement of the microrobot 10 in fluidic materials with a low Reynolds number, such as the cerebrospinal fluid or the extracellular matrix of the brain.

The propulsion and steering device 1 also comprises a control unit 9 configured to actuate, by selectively controlling one or more of the electrical connections 83, 85, 87, a rotation of the propulsion element 2 about at least one axis transverse to the main axis X₂. The control unit 9 is also configured to actuate a deformation of the deformable portion 20 in elongation/contraction according to the main axis X₂. Thus, it is possible to optimally coordinate the actuation of the rotation of the propulsion element 2 and the actuation of the deformation in elongation/contraction of the deformable portion 20 according to the main axis X₂ in order to obtain desired movement and trajectory of the microrobot 10 in the material in which it moves. The control unit 9 thus actuates a single element, the propulsion element 2, and allows generating, by the actuation of this single element, a propulsion and a rotation of the device 1.

A method for propelling and steering the microrobot 10 in a fluid with a low Reynolds number, in particular comprised between 10⁻⁵ and 10⁻¹, comprises the selective control of one or more of the electrical connections 83, 85, 87, using the control unit 9, to actuate a rotation of the propulsion element 2 about at least one axis transverse to the main axis X₂ in a coordinated manner, whether simultaneously or sequentially, with a deformation of the deformable portion 20 in elongation/contraction according to the main axis X₂.

As a non-limiting example, a microrobot 10 having the following characteristics has good propulsion and guidance performances in fluidic materials with a low Reynolds number:
total length of the microrobot 10: 2 mm;
diameter of the microrobot 10: 2 mm;
length of the deformable portion 20 of the propulsion element 2: 0.5 mm;
length of the linear actuation coil 42: 0.5 mm;
length of the magnet 41: 0.8 mm;
cross-section of each propulsion cilium 28: 2,500 μm².

Manufacturing Process

The front portion 21, the rear portion 23 and the deformable portion 20 have been manufactured in one piece by 3D laser lithography using a UV-curable hybrid inorganic-organic ORMOCLEAR polymer as a photoresist. The photoresist has been applied on a glass substrate and a point laser has selectively cured the photoresist according to a 3D CAD plan. The propulsion cilia 28 have been made in one piece with the front portion 21, i.e. manufactured in the same material as the front portion 21. The guide segments 3, 5, 7 have been obtained by deposition of a layer of a PEDOT ionic electroactive polymer on each of the flexible legs 22, 24, 26 of the deformable portion 20. The linear actuation coil 42 has been obtained by winding a copper wire on the rear portion 23. The magnet 41 is a neodymium permanent magnet fastened by gluing with an acrylic adhesive on the front portion 21.

In the second embodiment represented in FIG. 4, elements similar to those of the first embodiment bear identical references. The microrobot 10 of the second embodiment differs from the first embodiment in that the guide segments 3, 5, 7 include a photoactive material, instead of an electroactive material. The propulsion and steering device 1 comprises, for each guide segment 3, 5, 7 based on a photoactive material, a dedicated radiation source whose radiation is brought opposite the guide segment so as to activate the deformation thereof. As example, in this second embodiment, the photoactive material of each guide segment 3, 5, 7 is a network of liquid crystals comprising azobenzene molecules, and the radiation source for each guide segment 3, 5, 7 is a white light source, the different sources being housed within the same case 8′.

In this second embodiment, all guide segments 3, 5, 7 are based on the same photoactive material and, to avoid radiation interactions likely to activate the deformation of a guide segment other than that associated to a dedicated radiation source, the radiation is transmitted up to the photoactive material of each guide segment 3, 5, 7 using a respective optical fibre 83′, 85′, 87′, having a distal end positioned opposite the photoactive material of the guide segment 3, 5, 7. According to one variant, the guide segments 3, 5, 7 may be based on different photoactive materials, adapted to be activated by radiations of different wavelengths. In this case, each guide segment 3, 5, 7 is associated to a radiation source emitting in the wavelength range that is specific to it. Herein again, the radiation could be transmitted up to the photoactive material of the guide segment 3, 5, 7 using an optical fibre having a distal end positioned opposite the photoactive material of the guide segment.

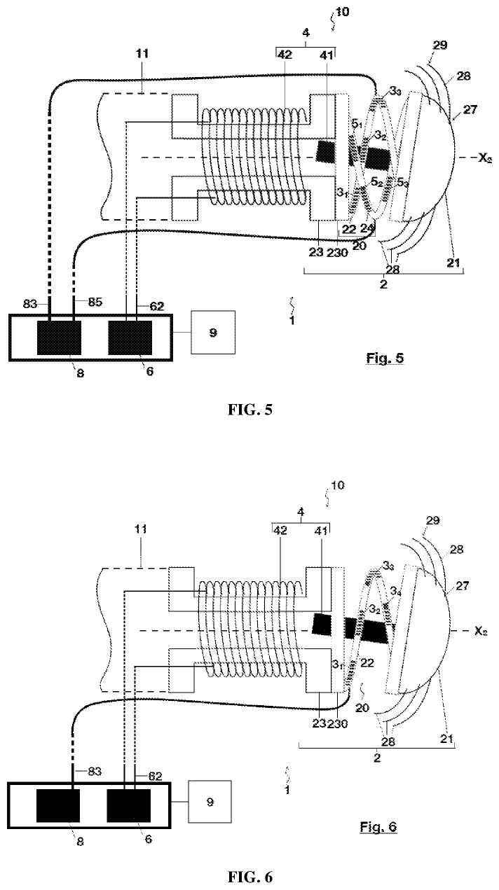

In the third embodiment represented in FIG. 5, elements similar to those of the first embodiment bear identical references. The microrobot 10 of the third embodiment differs from the first embodiment in that the deformable portion 20 of the propulsion element 2 is a helical spring comprising two flexible legs 22, 24, instead of three flexible legs like in the first embodiment. The two flexible legs 22, 24 are helically disposed around the main axis X₂ between the front portion 21 and the rear portion 23 of the propulsion element and each is provided with three guide segments based on an electroactive material, respectively 3₁, 3₂, 3₃ and 5₁, 5₂, 5₃. For each of the two flexible legs 22, 24, the guide segments 3₁, 3₂, 3₃ or 5₁, 5₂, 5₃ are distributed along the flexible leg and connected to an electrical power supply 8 by a respective electrical wire, all electric wires of the different guide segments of a flexible leg 22 or 24 transiting in a cable 83 or 85.

In the fourth embodiment represented in FIG. 6, elements similar to those of the first embodiment bear identical references. The microrobot 10 of the fourth embodiment differs from the first embodiment in that the deformable portion 20 of the propulsion element 2 is a helical spring comprising a unique flexible leg 22 helically disposed around the main axis X₂ between the front portion 21 and the rear portion 23 of the propulsion element. The flexible leg 22 includes four guide segments based on an electroactive material 3₁, 3₂, 3₃, 3₄, which are distributed along the flexible leg 22 and each connected to an electrical power supply 8 by a respective electrical wire, all electric wires of the different guide segments of the flexible leg 22 transiting in a cable 83. The guide segments 3₁, 3₂, 3₃, 3₄ are configured to generate by their deformation a rotation of the propulsion element 2 respectively about a first axis of rotation and about a second axis of rotation transverse to each other and to the main axis X₂ of the propulsion element.

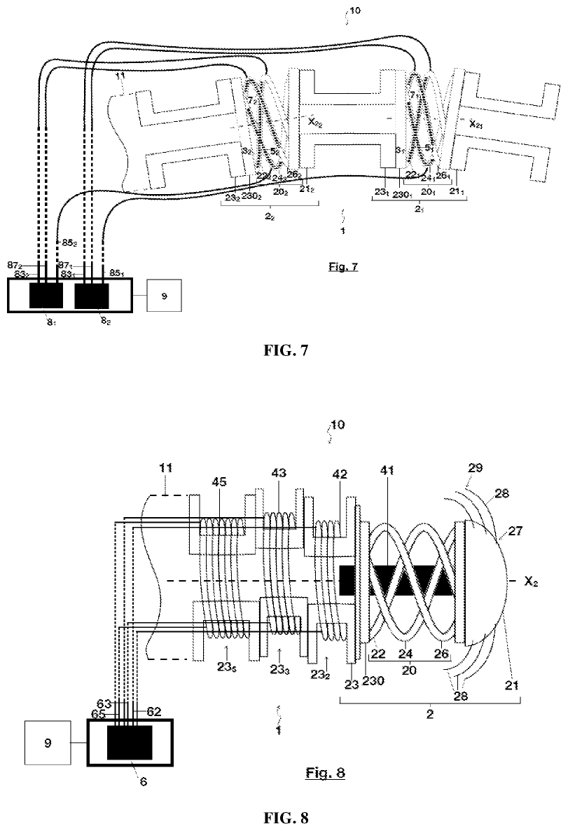

In the fifth embodiment represented in FIG. 7, elements similar to those of the first embodiment bear identical references. The microrobot 10 of the fifth embodiment differs from the first embodiment in that the propulsion and steering device 1 comprises two propulsion elements 2₁ and 2₂ arranged one behind another, the control unit 9 being configured to actuate elongation/contraction deformation cycles of the deformable portions 20₁ and 20₂ of the two propulsion elements according to predefined time sequences, so as to generate a non-reciprocal movement of the microrobot 10. Such an arrangement is a means other than propulsion cilia to achieve a non-reciprocal movement of the microrobot 10, allowing for an efficient movement in fluids with a low Reynolds number.

In this fifth embodiment, for each of the two propulsion elements 2₁ and 2₂, the deformable portion 20₁ or 20₂ is identical to the deformable portion 20 of the first embodiment, i.e. comprises three flexible legs 22₁, 24₁, 26₁ or 22₂, 24₂, 26₂ helically disposed around the main axis X₂₁ or X₂₂ of the propulsion element. Each flexible leg 22₁, 24₁, 26₁ or 22₂, 24₂, 26₂ is provided with a respective guide segment 3₁, 5₁, 7₁ or 3₂, 5₂, 7₂ based on an electroactive material, reversibly deformable under the effect of an electrical energy supply, and connected to an electrical power supply 8₁ or 8₂ by a respective electrical cable 83₁, 85₁, 87₁ or 83₂, 85₂, 87₂.

The propulsion and steering device 1 of this fifth embodiment does not comprise a linear actuator similar to the electromagnetic transducer 4 of the previous embodiments, to sequentially actuate elongation/contraction cycles of the deformable portion 20₁ or 20₂ of the propulsion element. Indeed, in this fifth embodiment, for each of the two propulsion elements 2₁ and 2₂, the guide segments 3₁, 5₁, 7₁ or 3₂, 5₂, 7₂ based on an electroactive material are configured to actuate a deformation of the deformable portion 20₁ or 20₂ in elongation/contraction according to the main axis X₂₁ or X₂₂ when they are deformed simultaneously, and to actuate a rotation of the propulsion element 2₁ or 2₂ about an axis of rotation transverse to the main axis X₂₁ or X₂₂ when they are selectively deformed. By supplying electrical energy selectively to the guide segments 3₁, 5₁, 7₁, 3₂, 5₂, 7₂, it is possible to actuate a rotation and/or a deformation in elongation/contraction of each propulsion element 2₁, 2₂, which allows ensuring both directional steering and propulsion of the microrobot 10.

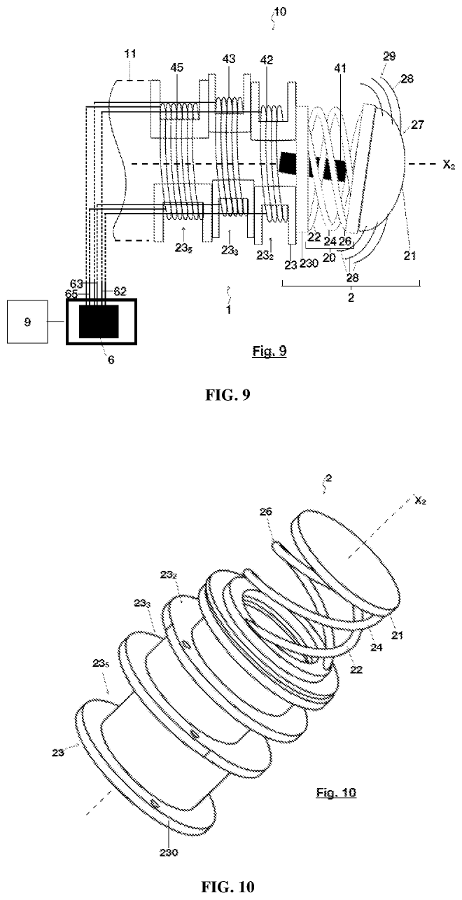

In the sixth embodiment represented in FIGS. 8 to 10, elements similar to those of the first embodiment bear identical references. The microrobot 10 of the sixth embodiment differs from the first embodiment in that the guide elements comprise two electromagnetic guide coils 43 and 45, instead of guide segments based on an active material. Each of the guide coils 43 and 45 is provided with a respective connection 63, 65 to an electrical energy source 6 and forms an electromagnetic transducer with a permanent magnet 41 secured to the front portion 21 of the propulsion element 2. The magnet 41 is substantially parallel to the main axis X₂ of the propulsion element in the rest position. Each of the two guide coils 43, 45 is adapted to generate, under the effect of an electrical energy supply, a rotation of the magnet 41 with respect to its rest position, which causes a rotation of the propulsion element 2 about an axis of rotation transverse to the main axis X₂.

The propulsion and steering device 1 of this sixth embodiment also comprises a linear actuation electromagnetic coil 42, similar to the coil 42 of the previous embodiments, which is provided with a respective connection 62 to the electrical energy source 6 and which also forms an electromagnetic transducer with the magnet 41. The linear actuation coil 42 is adapted to generate, under the effect of an electrical energy supply, a translation of the magnet 41 parallel to the main axis X₂, which causes a deformation of the deformable portion 20 in elongation/contraction according to the main axis X₂. By selectively powering the guide coils 43, 45 and the linear actuation coil 42 electrically, it is then possible to actuate a rotation and a deformation in elongation/contraction of the propulsion element 2, which allows ensuring directional steering and propulsion of the microrobot 10.

The relative arrangement of the linear actuation coil 42 and the guide coils 43, 45 is shown in the larger scale view of FIG. 10. This figure shows the respective grooves 23₂, 23₃, 23₅ for receiving the coils 42, 43, 45. The central axis of the linear actuation coil 42 received in the groove 23₂ is aligned with the main axis X₂ of the propulsion element 2. The central axis of the guide coil 43 received in the groove 23₃ is offset with respect to the main axis X₂ of the propulsion element 2, according to an upward direction and a direction extending into the plane of the sheet in FIG. 10. Finally, the central axis of the guide coil 45 received in the groove 23₅ is offset with respect to the main axis X₂ of the propulsion element 2, according to a downward direction and a direction emerging from the plane of the sheet in FIG. 10.

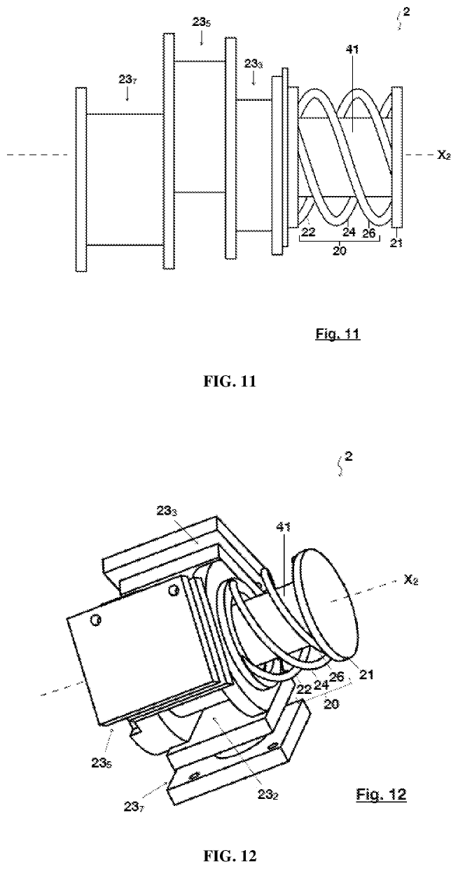

In the seventh embodiment represented in FIG. 11, elements similar to those of the sixth embodiment bear identical references. In this seventh embodiment, the propulsion and steering device 1 comprises three guide coils 43, 45, 47 (not represented) each provided with a respective connection to an electrical energy source and configured to form an electromagnetic transducer with a permanent magnet 41 secured to the front portion of the propulsion element 2. In FIG. 11, the respective grooves 23₃, 23₅, 23₇ for receiving the guide coils 43, 45, 47 are shown. The three guide coils 43, 45, 47 are disposed one behind another according to the direction of the main axis X₂ of the propulsion element 2, with their central axes not coincident with and substantially parallel to the main axis X₂.

In particular, in the example represented in FIG. 11, the central axis of the guide coil 43 received in the groove 23₃ is offset with respect to the main axis X₂ of the propulsion element 2, according to a downward direction and extending into the plane of the sheet in FIG. 11. The central axis of the guide coil 45 received in the groove 23₅ is offset with respect to the main axis X₂ of the propulsion element 2, according to an upward direction and extending into the plane of the sheet in FIG. 11. Finally, the central axis of the guide coil 47 received in the groove 23₇ is offset with respect to the main axis X₂ of the propulsion element 2, according to a downward direction and a direction emerging from the plane of the sheet in FIG. 11. The three guide coils 43, 45, 47 are configured to actuate a deformation of the deformable portion 20 of the propulsion element 2 in elongation/contraction according to the main axis X₂ when they are simultaneously powered with electrical energy, and to generate a rotation of the magnet 41 with respect to its rest position causing a rotation of the propulsion element 2 about an axis of rotation transverse to the main axis X₂ when they are selectively powered.

In the eighth embodiment represented in FIG. 12, elements similar to those of the sixth embodiment bear identical references. In this eighth embodiment, the propulsion and steering device 1 comprises a linear actuation coil 42 and three guide coils 43, 45, 47 (not represented), each provided with a respective connection to an electrical energy source and configured to form an electromagnetic transducer with a permanent magnet 41 secured to the front portion of the propulsion element 2. In FIG. 12, the respective grooves 23₂, 23₃, 23₅, 23₇ for receiving the coils 42, 43, 45, 47 are shown. The linear actuation coil 42 is arranged at the rear of the propulsion element 2 with its central axis substantially parallel to the main axis X₂ of the propulsion element 2, whereas the three guide coils 43, 45, 47 are distributed around the linear actuation coil 42 while being equidistant from each other, with their central axes substantially perpendicular to the main axis X₂.

In this eighth embodiment, the actuation of the deformation of the deformable portion 20 in elongation/contraction according to the main axis X₂ is obtained by powering the linear actuation coil 42, whereas the actuation of the rotation of the magnet 41 with respect to its rest position, causing a rotation of the propulsion element 2 about an axis of rotation transverse to the main axis X₂, is obtained by selectively powering the guide coils 43, 45, 47.

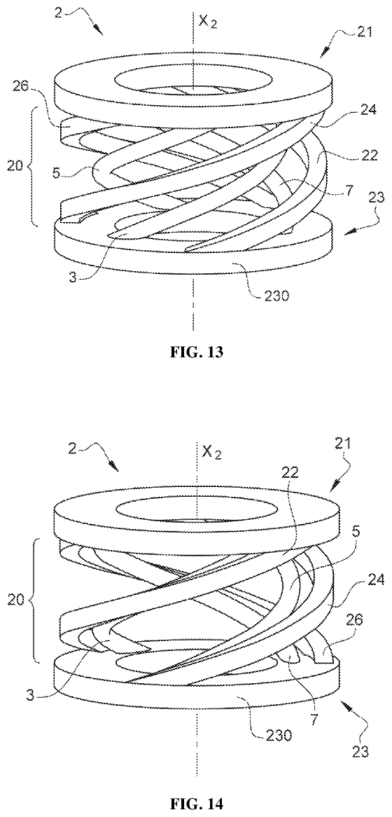

In the ninth and tenth embodiments represented respectively in FIGS. 13 and 14, elements similar to those of the sixth embodiment bear identical references. In these ninth and tenth embodiments,

The propulsion element 2 according to the ninth and tenth embodiments includes, like the propulsion element of the embodiment illustrated in FIG. 3, a front portion 21, a rear portion 23, and a deformable portion 20 connecting the front portion 21 and the rear portion 23. In these ninth and tenth embodiments, the deformable portion 20 is a helical spring deformable in elongation/contraction according to a main axis X₂ of the propulsion element 2. The axis X₂ is defined in the same manner as before, like the central axis of the deformable portion 20 substantially perpendicular to the plane of the distal plate 230 of the rear portion 23, to which the deformable portion 20 is fastened. The helical spring forming the deformable portion 20 comprises three flexible legs 22, 24, 26 helically disposed around the main axis X₂ between the front portion 21 and the rear portion 23 of the propulsion element 2.

In these ninth and tenth embodiments, the helical spring forming the deformable portion 20 cooperates with at least one guide element 3, 5, 7 each extending between the front portion 21 and the rear portion 23 of the propulsion element 2. In a non-represented embodiment, the guide elements extend around the helical spring. In the ninth and tenth embodiments, the helical spring extends around the at least one guide element 3, 4, 5. More particularly, in the ninth and tenth embodiments, the device 1 includes three guide elements 3, 5, 7 each forming a deformable leg or segment, helically disposed around the main axis X₂ between the front portion 21 and the rear portion 23 of the propulsion element. In the ninth embodiment illustrated in FIG. 13, the deformable segments 3, 5, 7 and the flexible legs 22, 24, 26 are evenly distributed over the circumference of the propulsion element 2, so that the propulsion element 2 has a circular alternation of flexible legs 22, 24, 26 and deformable segments 3, 5, 7. In the tenth embodiment illustrated in FIG. 14, each deformable segment 3, 5, 7 is radially aligned with a flexible leg 22, 24, 26 of the helical spring. Thus, each flexible leg 22, 24, 26 cooperates, in each of the embodiments of FIGS. 13, 14, with a guide element 3, 5, 7.

Like in the previous embodiments, each deformable segment 3, 5, 7 includes, for example, an electroactive material, for example a PEDOT ionic electroactive polymer. Thus, each of the three guide elements 3, 5, 7 is reversibly deformable under the effect of an electrical energy supply. Each guide element 3, 5, 7 is adapted to generate, by the deformation thereof when it is powered with electrical energy, a deformation of the corresponding flexible leg 22, 24, 26 and a rotation of the propulsion element 2. For each guide segment 3, 5, 7, the axis of the rotation generated by the deformation of the guide segment is transverse to the main axis X₂ of the propulsion element 2 as well as to the axes of the rotations generated by the deformation of each of the other two guide elements. The isotropic distribution of the guide segments 3, 5, 7 around the main axis X₂ of the propulsion element 2, allows, like in the first embodiment, optimising the directional steering of the propulsion element 2. Thus, regardless of the embodiment, it should be noticed that, in the present invention, the guide segments 3, 5, 7 form, with the flexible legs 22, 24, 26 a unique versatile functional group ensuring both rotation and propulsion. The present invention does not feature any coupling of different elements, each ensuring a distinct function.

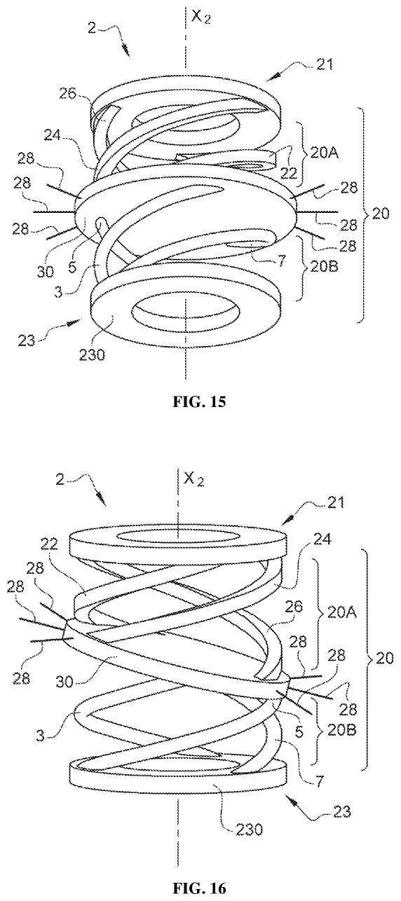

In the eleventh embodiment represented in FIGS. 15 and 16, elements similar to those of the first embodiment bear identical references. In this eleventh embodiment, the device 1 for propelling and steering the microrobot 10 is, like in the previous embodiments, configured to move in a viscous or viscoelastic material, for example in the cerebrospinal fluid or the extracellular matrix of a subject's brain, which are fluidic materials with a low Reynolds number.

In FIGS. 15 and 16, an alternative embodiment of the propulsion element 2 is represented. In this eleventh embodiment, the propulsion element 2 includes a front portion 21, a rear portion 23, and a deformable portion 20 connecting the front portion 21 and the rear portion 23. The deformable portion 20 is divided into a front sub-portion 20A and a rear sub-portion 20B, the two sub-portions 20A, 20B are connected together by an oscillating disc 30. The oscillating disc 30 is located between the front 21 and rear 23 portions, equidistant from each of these. In the example illustrated in FIGS. 15 and 16, the oscillating disc 30 has a diameter similar to the distal plate 230. Nonetheless, in a non-illustrated embodiment, the diameter of the oscillating disc 30 may be larger than that of the distal plate 230.

At rest, the oscillating disc 30 is substantially parallel to the distal plate 230. In this embodiment, it is the oscillating disc 30 of the propulsion element 2 which comprises, at its surface, a plurality of propulsion cilia 28, configured to interact with the material in which the microrobot 10 moves. The sequential elongation/contraction cycles of the deformable portion 20 cause the movement of the propulsion cilia 28 in the material, producing the propulsive force which causes a movement of the microrobot 10. Thus, it may be advantageous for the oscillating disc 30 to have a larger diameter than the distal plate 230, so as to facilitate the attachment of the propulsion cilia 28 thereon.

In this eleventh embodiment, the front sub-portion 20A of the deformable portion 20 is a helical spring deformable in elongation/contraction according to a main axis X₂ of the propulsion element 2. The main axis X₂ of the propulsion element 2 is herein defined in a manner similar to the previous embodiments, like the central axis of the deformable portion 20 substantially perpendicular to the plane of the distal plate 230 of the rear portion 23, to which the deformable portion 20 is fastened. The helical spring forming the front sub-portion 20A of the deformable portion 20 comprises three flexible legs 22, 24, 26 helically disposed around the main axis X₂ between the front portion 21 and the oscillating disc 30 of the propulsion element.

In this eleventh embodiment, the rear sub-portion 20B of the deformable portion 2 includes at least one guide element 3, 5, 7 based on an electroactive material, for example a PEDOT ionic electroactive polymer. More specifically, in the eleventh embodiment of the invention, the deformable portion 2 includes three guide elements 3, 5, 7 forming guide segments 3, 5, 7. Each of the three guide segments 3, 5, 7 is reversibly deformable by the effect of an electrical energy supply, and connected to an electrical power supply. At rest, the three guide segments 3, 5, 7 have the same length. As clearly shown in FIG. 15, the guide segments 3, 5, 7 are isotropically distributed around the main axis X₂ of the propulsion element 2, which allows optimising the directional steering of the propulsion element. Each of the three guide segments 3, 5, 7 forms a leg extending between the rear portion 23 of the deformable portion 2 and the oscillating disc 30. More specifically, the three guide segments are helically disposed around the main axis X₂ between the rear sub-portion 23 and the oscillating disc 30 of the propulsion element 2. As already mentioned, each guide segment 3, 5, 7 is adapted to generate, by the deformation thereof when it is powered with electrical energy, an inclination of the oscillating disc 30. This is shown in FIG. 16. As each of the three guide segments 3, 5, 7 is activated, the oscillating disc 30 tilts in different directions, thus generating a rotary oscillating movement. This rotary oscillating movement induces a rotation of the propulsion element 2. For each guide segment 3, 5, 7, the axis of the rotation generated by the deformation of the guide segment is transverse to the main axis X₂ of the propulsion element as well as to the axes of the rotations generated by the deformation of each of the other two guide segments. Thus, in this embodiment too, despite the presence of the oscillating disc 30, the guide segments 3, 5, 7 cooperate directly with the flexible legs 22, 24, 26 and form with these a unique versatile functional group ensuring both rotation and propulsion. The present invention does not feature any coupling of different elements, each ensuring a distinct function.

As it arises from the previous examples, a propulsion and steering device according to the invention allows moving a microstructure in the 3D space reliably and accurately by actuating, in a coordinated manner, on the one hand a rotation of the propulsion element about at least two axes of rotation transverse to each other and to the main axis, and on the other hand a deformation of the deformable portion of the propulsion element to generate a propulsion of the microstructure. Advantageously, thanks to the possibility of activating the energy supply independently for each guide element and possibly for the linear actuator if this is present, all spatial and temporal combinations for actuating the rotation and the deformation of the deformable portion of the propulsion element could be considered. In particular, the rotation and the deformation could, as desired, be actuated simultaneously, or be actuated one after another, which allows moving the microstructure according to a desired trajectory in its environment.

One should bear in mind that on a millimetre scale, in a low Reynolds environment, the smallest element to be moved requires a lot of energy. The involved frictional forces are considerable. Although depending on the type of frictions (dry, viscous, etc.) and on the size of the robot, it is nonetheless known that, in general, a low Reynolds number means that the surface forces are predominant compared to the volumetric forces. In this case, it is thus more appropriate to optimise the overall size of the robot than its weight, for example.

Thus, the smaller the device and the fewer functional elements it includes, the lower the energy expense to move said device will be. Thanks to its small size and the reduced number of functional elements (reduction made possible by the polyfunctional aspect of the different elements, in particular the guide segments), the present invention allows for a significant energy saving for a given movement.

The invention is not limited to the described and represented examples.

In particular, in the previous examples, the deformable portion of the propulsion element is a helical spring with one, two or three flexible leg(s). Alternatively, the deformable portion of the propulsion element may comprise a spring, helical or not, having any number of flexible legs, or else a deformable structure other than a spring, for example a bellow. The deformable portion of the propulsion element may also comprise a combination of a spring and a bellow, each fold of the bellow being for example positioned at a turn of the spring and the envelope of the bellow filling the space between the successive turns of the spring.

In addition, in the case where the propulsion and steering device comprises a dedicated linear actuator for actuating the deformation of the deformable portion of the propulsion element in elongation/contraction, the linear actuator may be an actuator other than an electromagnetic transducer as previously described, involving an electromagnetic coil and a permanent magnet. In particular, in the case where the deformable portion has a sealed envelope, as is the case for example with a bellow, the actuator for actuating the deformation of the deformable portion in elongation/contraction may be a pump, the elongation/contraction of the deformable portion could then be obtained through an alternation of inflows/outflows of fluid in the internal volume of the deformable portion actuated by the pump.

Moreover, in the previous examples implementing guide segments comprising an active material, the active materials of the different guide segments are all of the same nature. Alternatively, a propulsion and steering device according to the invention may include several guide segments having active materials of different compositions or natures. For example, guide segments including an electroactive material may be combined with guide segments including a bimetallic element; or guide segments including a photoactive material may be combined with guide segments including an electroactive material or a bimetallic element, the different energy supply connections for activating the guide segments being adapted accordingly. Guide segments based on an active material may also be combined with guide coils of the type of those of the embodiments of FIGS. 8 to 12.

In the case where the propulsion and steering device comprises guide coils as guide elements for generating a rotation of the propulsion element, arrangements of the guide coils other than those of the embodiments of FIGS. 8 to 12 could of course also be considered. In particular, the number of guide coils is any number greater than or equal to two, the guide coils being possibly arranged one behind another, one next to another, or even concentrically, while being combined, or not, with a linear actuation coil.

Advantageous arrangements, not represented in the figures, comprise for example: three guide coils distributed around the main axis of the propulsion element, with their central axes substantially parallel to the main axis, while being disposed equidistant from each other; six guide coils distributed around the main axis of the propulsion element, with their central axes substantially parallel to the main axis, while being disposed equidistant from each other. In these two cases, the guide coils could be disposed, as desired, either at the rear of the propulsion element without a linear actuation coil, the actuation of the deformation of the deformable portion in elongation/contraction according to the main axis then being obtained by simultaneously powering all guide coils with electrical energy, whereas the actuation of the rotation of the magnet with respect to its rest position, causing a rotation of the propulsion element about an axis of rotation transverse to the main axis, is obtained by selectively powering the guide coils; disposed either at the rear of the propulsion element while being combined with a linear actuation coil, the actuation of the deformation of the deformable portion in elongation/contraction according to the main axis then being obtained by powering the linear actuation coil, whereas the actuation of the rotation of the magnet with respect to its rest position, causing a rotation of the propulsion element about an axis of rotation transverse to the main axis, is obtained by selectively powering the guide coils.

Finally, the invention has been illustrated for the propulsion and steering of a microrobot intended to move in a viscous or viscoelastic material, for example the cerebrospinal fluid or the extracellular matrix of a subject's brain. Alternatively, a propulsion and steering device according to the invention could of course be implemented to move other types of microstructures, in the medical field or in other fields, in particular a device according to the invention could be used for the propulsion and steering of a movable flexible tube, such as a stent or a catheter.

1-11. (canceled)

12. A device for propelling and steering a microstructure, such as a flexible tube or a microrobot, comprising:
a propulsion element including at least one portion deformable in elongation/contraction according to a main axis connecting a front portion and a rear portion of the propulsion element;
at least two guide elements adapted to generate, under the effect of an energy supply by a respective connection to an energy source, a rotation of the propulsion element respectively about a first axis of rotation and about a second axis of rotation transverse to each other and to the main axis of the propulsion element;
a control unit configured to actuate, by selectively controlling one or more of the connections to an energy source, a rotation of the propulsion element about at least one axis transverse to the main axis in a coordinated manner with a deformation of the deformable portion of the propulsion element in elongation/contraction according to the main axis, the guide elements further comprising at least two guide segments based on an active material reversibly deformable under the effect of an energy supply by a respective connection to an energy source, each guide segment being adapted to generate by the deformation thereof, under the effect of an energy supply, a rotation of the propulsion element about an axis of rotation transverse to the main axis of the propulsion element.

13. The propulsion and steering device according to claim 12, wherein at least one guide segment includes an electroactive material or a bimetallic element, the device comprising an electrical energy source connected to the guide segment so as to activate the deformation thereof.

14. The propulsion and steering device according to claim 12, wherein at least one guide segment includes a photoactive material, the device comprising a radiation source whose radiation is emitted opposite the guide segment so as to activate the deformation thereof.

15. The propulsion and steering device according to claim 12, wherein at least two of said guide segments are configured to actuate a deformation of the deformable portion of the propulsion element in elongation/contraction according to the main axis when they are deformed simultaneously and to actuate a rotation of the propulsion element about an axis of rotation transverse to the main axis when they are selectively deformed.

16. The propulsion and steering device according to claim 12, wherein the guide elements comprise at least two electromagnetic guide coils, each provided with a respective connection to an electrical energy source, which form an electromagnetic transducer with a magnet secured to the propulsion element, the magnet being parallel to the main axis of the propulsion element in the rest position, each guide coil being adapted to generate, under the effect of an electrical energy supply, a rotation of the magnet with respect to its rest position causing a rotation of the propulsion element about an axis of rotation transverse to the main axis of the propulsion element.

17. The propulsion and steering device according to claim 16, further comprising a linear actuation electromagnetic coil, provided with a respective connection to an electrical energy source, which also forms an electromagnetic transducer with the magnet secured to the propulsion element, the linear actuation coil being adapted to generate, under the effect of an electrical energy supply, a translation of the magnet parallel to the main axis causing deformation of the deformable portion of the propulsion element in elongation/contraction according to the main axis.

18. The propulsion and steering device according to claim 12, wherein the control unit is further configured to actuate a deformation of the deformable portion of the propulsion element by elongation/contraction according to the main axis.

19. The propulsion and steering device according to claim 12, comprising an actuator, such as an electromagnetic transducer or a pump, configured to actuate a deformation of the deformable portion of the propulsion element in elongation/contraction according to the main axis.

20. The propulsion and steering device according to claim 12, wherein the at least two guide elements are positioned radially outside the deformable portion.

21. The propulsion and steering device according to claim 12, wherein that the deformable portion includes an oscillating disc disposed between the front portion and the rear portion, the at least two guide elements being disposed between the rear portion and the oscillating disc.

22. A method for propelling and steering a microstructure, such as a flexible tube or a microrobot, wherein:
the microstructure is introduced comprising a propulsion and steering device according to claim 12 in a fluid with a low Reynolds number comprised between 10⁻⁵ and 10⁻¹;
one or more of the connections to an energy source are actuated, by selectively controlling using the control unit, a rotation of the propulsion element about at least one axis transverse to the main axis in a coordinated manner with a deformation of the deformable portion of the propulsion element in elongation/contraction according to the axis main.
